(19) United States
(12) Patent Application Publication
(10) Pub. No.: US 2005/0042301 A1
(43) Pub. Date: Feb. 24, 2005

(54) Method of use of Xenon-133 for the treatment of AIDS, other viral and non-viral infections

(76) Inventor: Donald R. VanDeripe, St. Charles, MO (US)

Correspondence Address: Donald R. VanDeripe, 1534 Woodbury Drive, St. Charles, MO, 63304, US

(21) Appl. No.: 10/642,540

(22) Filed: Aug. 18, 2003

(51) Int. Cl. A61K 33/00

(52) U.S. Cl. 424/600

(57) ABSTRACT

The disclosure describes a method of use of the radioisotope Xenon-133 for the treatment of AIDS, other viral and non-viral infections. The treatment entails the inhalation of a gas mixture containing the radioactive, beta-particle emitting Xe-133 gas for providing superior targeting and destruction of the AIDS vectors in the blood, lymph and body water when compared against external beam X-ray therapy.

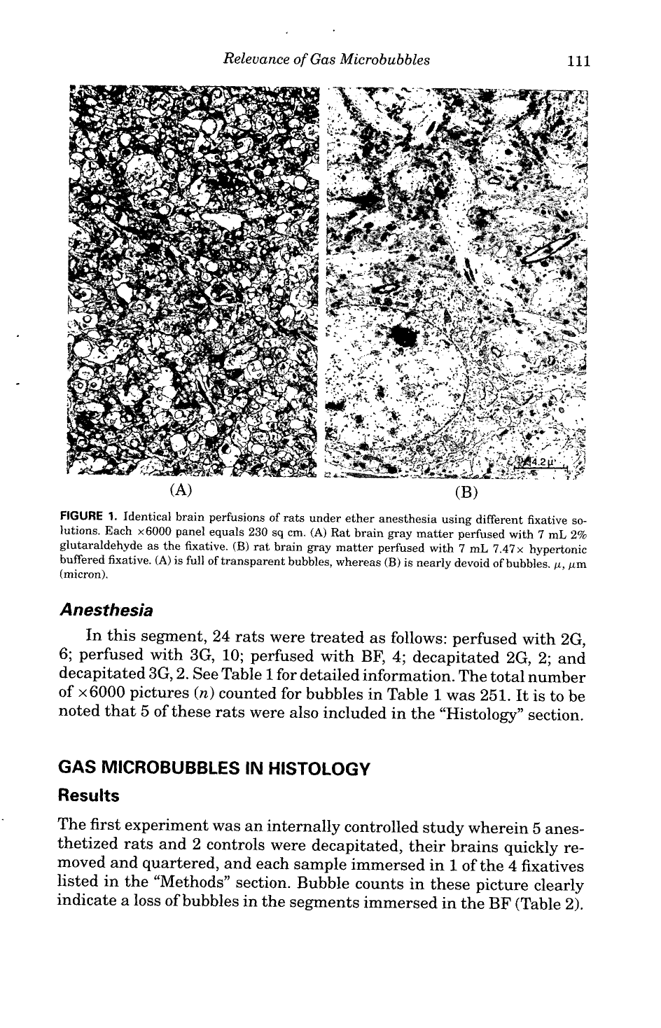

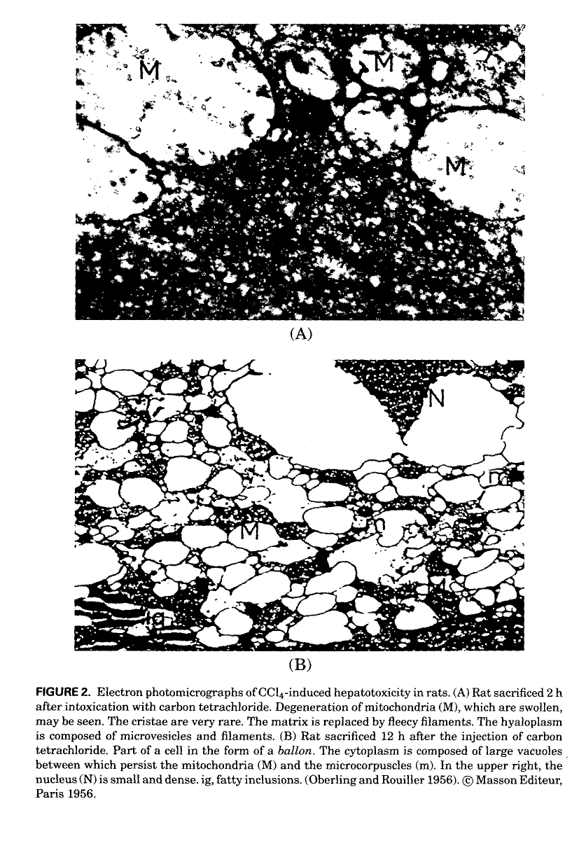

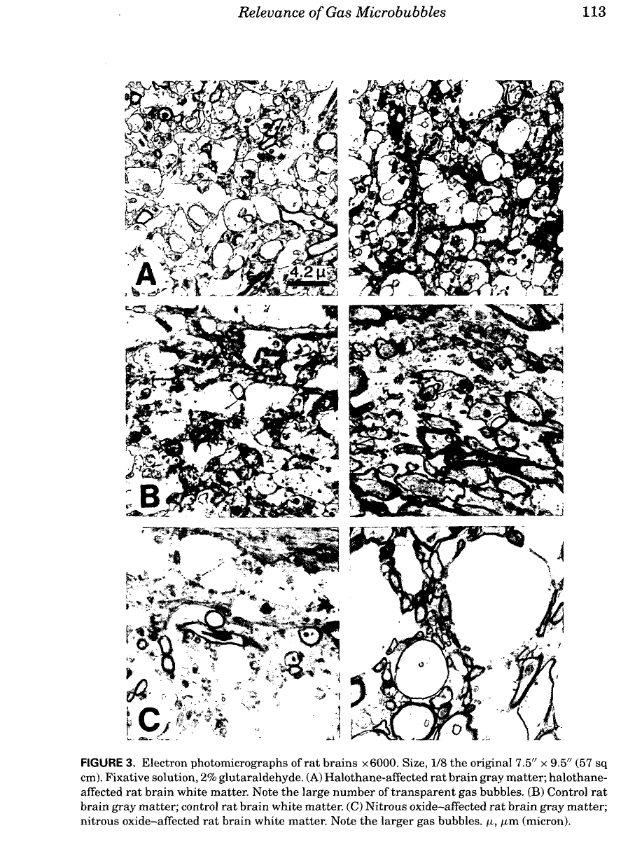

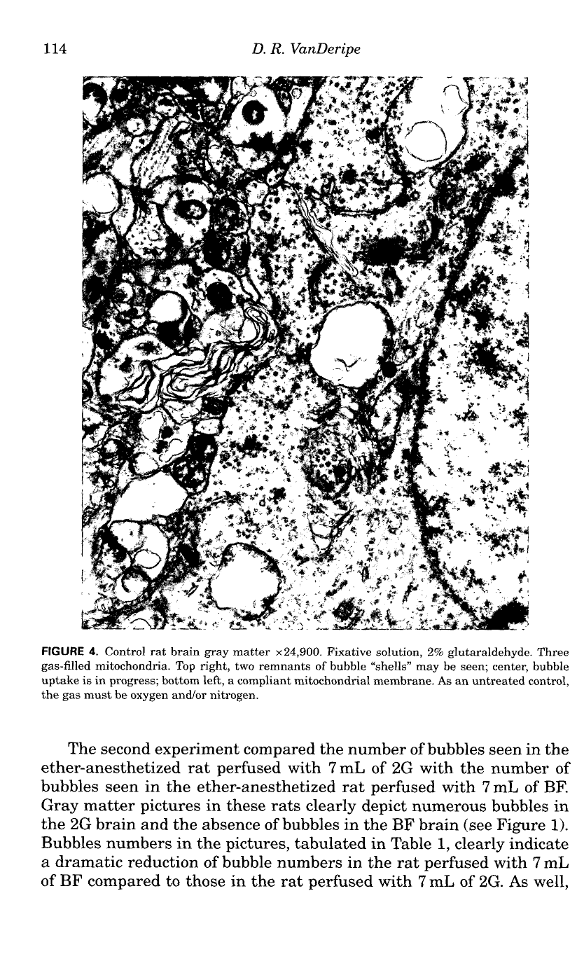

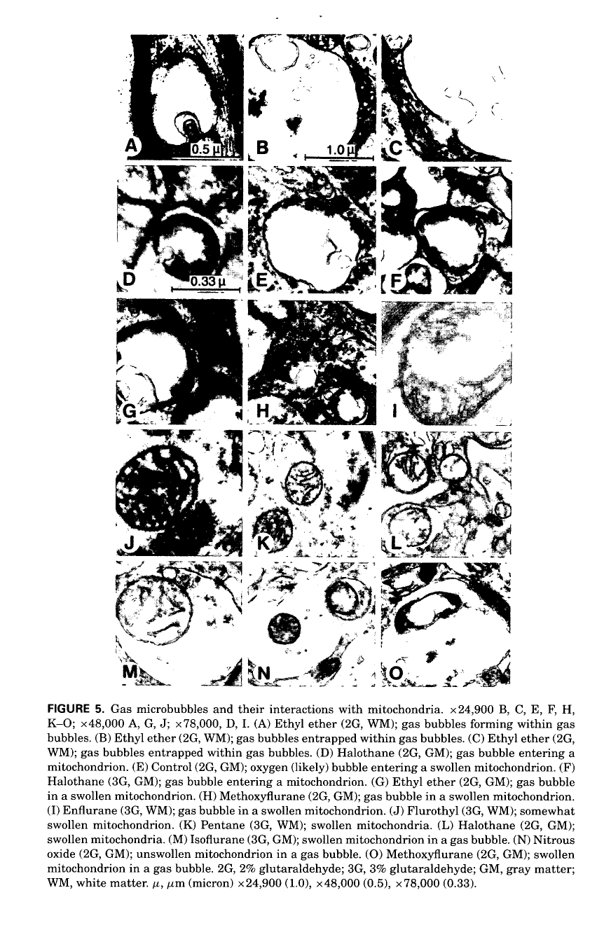

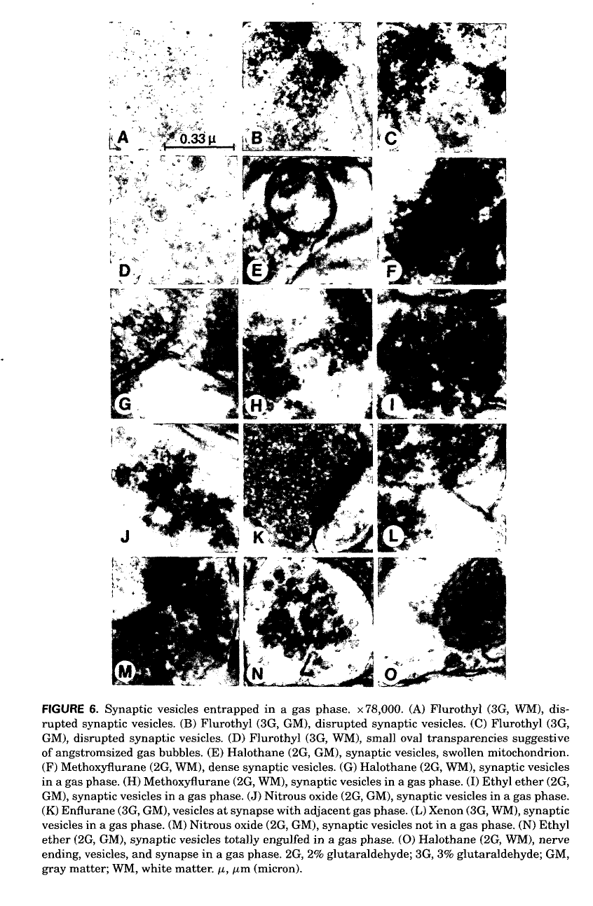

### CROSS REFERENCE TO RELATED APPLICATIONS

Disclosure Document Number 497172

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

There is a great need for treatments which can cure AIDS, other viral diseases and certain non-viral infections. Generally speaking, drug therapy treatments for viral diseases are only somewhat effective at controlling the proliferation of viral DNA. Moreover, prion based diseases are essentially untreatable and antibiotic resistant bacteria are becoming an ever increasing problem.

Attempts to kill the AIDS virus with various external beam X-ray treatments have met with mixed results. Bigbee, et al, [Inactivation of Human Immunodeficiency virus (HIV) by Ionizing Radiation in Body Fluids and Serological Evidence; Journal of Forensic Sciences, Vol 34, pp 1303-1310, 1989] studied the inactivation of the AIDS virus in the laboratory and found that 25,000 rads of radiation were required to kill the virus in vitro when present at concentrations of 50 million copies per milliliter. Since a dose of about 1000 rads of ionizing radiation is lethal to humans [Von Sonntag, The Chemical Basis of Radiation Biology, Taylor and Francis, New York, 1987], administering a radiation dose of 25,000 rads to humans would be out of the question. Interestingly, three studies in mice have demonstrated an enhanced anti-viral effect when Friend Leukemia Virus (FLV) was inoculated into mice. [Shen et al, J. Radiation Oncology Biol Phys v 16, pp 165-170, 1989; Adv Exp Med Biol v 407, pp 451-458 1997; and “in vivo” v 10, pp 191-200, 1996]. The in vitro exposure of the FLV, a mouse equivalent to human AIDS, to 1000 rads of x-irradiation prior to inoculation into mice did not decrease its infectivity. Nonetheless, Shen was able to demonstrate that as little as 150 rads of external beam total body irradiation (TBI) of mice on days 5 and 12 following the FLV inoculation prolonged their life span to over one year, the same as untreated mice. Conversely, untreated inoculated mice died within 40 days. These studies demonstrated three potential enhancements over the in vitro protocol of Bigbee. 1) The number of viral particles per milliliter of blood in human AIDS is about 200, i.e. far less than the 50 million per ml of Bigbee. 2) Split TBI doses, i.e. repeat treatments, have value in treating in FLV viral infections. 3) The intrinsic body defense systems may be expected to augment the efficacy of radiation treatments. Regarding this last point, Shen et al showed that TBI of the FLV inoculated mice restored the levels of IFN-gamma, IL-2 and P53, diminished the levels of suppressor T-cells and increased the Natural Killer cell activity. This indicates that the TBI (150 rads, two exposures) did not act primarily by inactivating the viral DNA per se, but that the primary effect of the irradiation was to destabilize the infectious process, most likely by destroying the suppressor T cells and/or enhancing the production of cytokine (IFN-gamma, IL-2, P53) and other protective mediators. Faced with a major reduction in the blood viral load, the body's natural defenses were allowed to regenerate and successfully defeat the infection.

Based on these promising results, Shen suggested that clinical studies on TBI of AIDS patients should be considered and early efforts [del Regato, Am J. Clin Oncol v 12 p 365, 1989] were undertaken. Unfortunately, nothing further has been published, which leads one to conclude that the TBI was ineffective in humans, or, more likely that one or two 150 rad TBI doses was just not well tolerated by AIDS patients.

### BRIEF SUMMARY OF THE INVENTION

This inventor has conceived four additional steps beyond the therapy described by Shen, which should permit a 10-1000 fold reduction in the radiation dose required for effective treatment of FLV and presumably human AIDS, as well. These are:

a) Oxygenation of some tumors has been shown to enhance their radiosensitivity. Therefore, inhaling oxygen at concentration of 20%-95% is herein recommended as a part of the treatment described below.

b) Administration of the tissue irradiation within the body per se would be expected to provide for a major enhancement in efficacy since one could employ highly effective beta irradiation as opposed to the very inefficient external X-rays. Whereas X-rays are poorly absorbed by the body tissues and mainly result in glancing blows to the target, weak beta irradiations which travel only short distances before total absorption by the tissue can deliver 100% of their energy in vivo and much of that directly on the target.

c) Administration of the irradiating species in the form of a gas would assure its wide distribution in the blood, lymph and body water and it would be excreted via the lungs, thereby requiring no metabolic handling by the liver or kidneys.

d) Further optimizing the distribution of a radioactive gas would result from the admixture of 0.1-10% of the non-radioactive stable gas to the inhaled gas mixture.

The efficacy demonstrated by Shen et al can assuredly be enhanced from perhaps 10 to 100 and perhaps even 1000 fold by adding in factors a through d above to the treatment protocol. The ideal agent would consist of an inhaled radioactive beta-emitting isotope of Xenon, specifically Xenon133 (Xe-133), which has been safely used for years as a diagnostic radiopharmaceutical. Since it is produced in nuclear reactors, not cyclotrons, the supply of Xe-133 would be plentiful. It's radioactive half-life exceeds 5 days, which allows adequate time for production, distribution and clinical use. It's radioactive profile includes an 81 KeV gamma ray which is 38% abundant and which provides for its current clinical use as a lung scanning agent in Nuclear Medicine. More importantly, It also emits a series of weak beta particles which range in tissue penetration from about 5 microns (Auger electron) to about 1 millimeter. This beta emission profile assures a high linear energy transfer to tissues (fluids) in immediate contact with the Xe-133 gas. This inventor has demonstrated that stable Xenon-131 (Xe-131) is distributed in the body in the form of microbubbles of gas in a size range from about 5-10 microns in diameter [Gas Microbubbles in Biology: Their Relevance in Histology, Toxicology, Physiology and Anesthesia; Donald R. VanDeripe, Toxicology Methods, 11, 107-126, 2001]. It should be noted that the main enhancement in activity from the internally inhaled Xe-133 gas stems from the fact that the beta particles give up all their energy over short distances so that suppressor T cells, leukocytes and other biological targets may be destroyed by a single hit, whereas the likelihood of that occurrence from externally beamed X-rays would be rare since most of them traverse completely through the body. It is this amplification from beta particles which would provide for most of the improved efficacy from the inhaled Xe-133, however, it's administration as an inhaled gas and it's enhanced distribution by the admixture of Xe-131 also adds to the therapy.

### DETAILED DESCRIPTION OF THE INVENTION

It is proposed that the inhalation of radioactive xenon in the form of Xe-133, optionally mixed with low levels 0.1-10% of stable Xe-131 to enhance biodistribution will provide a useful radiotherapy for the treatment and/or cure of FLV, human AIDS, leukemias, and other retroviral diseases. For purposes of this disclosure the term—inhalation treatment—shall mean the complete process which includes the inhalation of a gas mixture through a mask from and into a closed system wherein re-breathing of said gas mixture continues for time sufficient to attain a specific exposure to Xe-133, and after which the mask is removed and the patient exhales into an ambient atmosphere or chamber, thereby excreting the tissue-residual inhaled gases from the body.

In practice, it is envisioned that patients would receive an inhalation treatment with a mixture of gases over a suitable time period to provide whole body radiation doses of up to about 25 rads maximum, such doses being administered in one-three divided treatments. Most favorably, the total dose would be substantially less (˜0.1-10 rads) and could be administered as a single treatment. The non-radioactive inhaled gases could include air, nitrogen, oxygen and stable Xe-131, whereas the radioactive gas would always be Xe-133. The relative concentration of xenon-131 would be optimized based on clinical use experience, but would typically range from about 0.1-10%. The oxygen content of the inhaled gas mixture would range from 20% up to about 95%, with any remainder being nitrogen or air. The inhaled Xe-133 would be admixed with the non-radioactive gas mixture and it's specific activity adjusted so that the desired TBI could be administered during a period of breathing, re-breathing and an exhalation (excretion) washout period. Note that with prolonged re-breathing procedures, a means for scrubbing carbon dioxide from the system might be required. Such methods are available in the art, having been employed in general anesthesia procedures for a number of years. Similarly, the gas masks, gas mixing equipment, and inhalation systems required for the therapy have been widely used in general anesthesia and nuclear medicine procedures. The total body irradiation (TBI) dose would be calculated based on an even distribution within the body water. For purposes of Pro Forma dosage calculations the MIRD dose tables list the radiation from the Xe-133 beta rays as being 2.77 rads per microcurie hour of exposure for a 100 milligram tissue sample. Assuming a 60 kg patient and 60% body water, there would be 360,000 tissue segments of 100 mg in weight. Therefore, 360 millicuries inhaled for one hour would expose the patient to 2.77 rads TBI, excluding the gamma ray component. If we arbitrarily ascribe 0.23 rads per hour to the xenon-133 gamma rays, then we approximate 3 rads per hour of TBI. Allowing 40 minutes post-inhalation for excretion from the body, another ˜2 rads might be projected for a total exposure of 5 rads. The exact dosimetry would vary with the kinetics of wash-in, steady state, and washout Xe-133 concentrations, and would be determined based on clinical use experience. For lower TBI doses of 1.5 and 0.15 rads, the total pulmonary mucosa target organ dose would recieve about 100 and 10 rads respectively, i.e. well within acceptable limits. For this to occur, the amplification in efficacy from inhaled Xe-133 over external beam X-ray therapy would have to approach 100 to 1000 fold which appears to be feasible considering that the beta emission profile of Xe-133 will assure a 100-1000 fold improvement in targeting due to the much greater tissue absorption of these radiations compared to external X-rays. One hundred to one thousand fold enhancements in efficacy could diminish the required radiation doses from Xe-133 to less than 5 rads, perhaps even to 0.15-1.5 rads permitting lower inhaled Xe-133 concentrations and shorter exposure times. A unique concept of the invention is the use of stable Xe-131 as a carrier gas for the radioactive Xe-133. This feature obtains from the work of the inventor cited above, wherein it was shown that inhaled Xe-131 forms microbubbles of gas in vitro and that these microbubbles range in size from about 5-10 microns in diameter. It is perceived that the Xe-133 will mix with the stable Xe-131 in these bubbles and in that way will be distributed throughout the blood, lymph and body water in a homogeneous fashion, therein assuring the effective radiation of circulating suppressor T cells, viral DNA and other AIDS-related causation factors.

The use of inhaled Xe-133 as a direct antiviral radiophramaceutical which denatures DNA and sub-DNA particles may prove useful for all viral, prion, and antibiotic resistant bacterial infections. Although the effective dose of TBI would be expected to be significantly higher than that required for AIDS, it would still be much lower than for any external beam radiation approach. For a direct anti-DNA effect, it is projected that doses exceeding 10 rads, and perhaps up to 50 rads might be required.

Modifications and extensions of this approach are to be expected in the literature and as such are to be considered as being anticipated by this disclosure. However, the inventor herein restricts his invention to the use of a single radioisotope as being acceptable for the therapeutic practice of this invention and that isotope is Xe-133.

1) In a warm blood animal, the method of single or multiple inhalation treatments employing Xenon-133 at millicurie concentrations and for a duration to expose the blood, lymph and body water and other organs and infected tissues of AIDS patients to absorbed radiation doses ranging from 0.1-10.0 rads, more preferably from about 0.1-2.0 rads, such radiatons being targeted primarily to the suppressor T cells and to the enhancement of natural body defense systems (INF-gamma, Le2, P53) and Natural Killer cells resulting in a decrease in circulating HIV virus particles (HIV-1, HIV-2, HTLV), therein providing a temporary relief from or permanent cure of the disease, and wherein the inhaled xenon-133 is admixed into a carrier gas formulation consisting of 20-95% oxygen, and optionally 0.1-10% Xenon-131, and with any remainder being air or nitrogen.

2) The claim as in 1, wherein the diseases treated are human leukemias.

3) In a warm blooded animal, the method of single or multiple inhalation treatments employing Xenon-133 at millicurie concentrations and for a duration to expose the blood, lymph, body water and other infected organs and tissues to absorbed radiation doses from 10-50 rads, such radiation being directed at viral or sub-viral DNA particles in viral, prion, or bacterial infections to effect a amelioration, reversal or cure of said infectious conditions.
